(19) United States
(12) Patent Application Publication
(10) Pub. No.: US 2024/0301388 A1
(43) Pub. Date: Sep. 12, 2024

(54) DELIGNIFIED BAMBOO SCAFFOLDS INCLUDING IMMOBILIZED ENZYMES AND METHODS FOR FORMING AND USING SAME

(71) Applicant: UNIVERSITY OF SOUTH CAROLINA, COLUMBIA, SC (US)

(72) Inventor: QIAN WANG, COLUMBIA, SC (US)

(21) Appl. No.: 18/597,113

(22) Filed: Mar. 6, 2024

Related U.S. Application Data
(60) Provisional application No. 63/489,264, filed on Mar. 9, 2023.

(51) Int. Cl. C12N 11/12; C12M 1/12

(52) U.S. Cl. CPC C12N 11/12; C12M 25/14

(57) ABSTRACT

Cellulose-based scaffolds for enzyme immobilization and methods for forming and using the scaffolds are described. The scaffolds incorporate delignified bamboo that can be functionalized to include an enzyme immobilized at a surface of the delignified bamboo via an alkylamine linkage. Disclosed scaffolds can be utilized in enzyme-based flow bioreactors.

### CROSS REFERENCE TO RELATED APPLICATION

This application claims filing benefit of U.S. Provisional Patent Application Ser. No. 63/489,264, having a filing date of Mar. 9, 2023, entitled “Enzyme Immobilization on Delignified Bamboo Scaffolds as a Multienzyme Bioreactor,” which is incorporated herein by reference for all purpose.

### FEDERAL RESEARCH STATEMENT

This invention was made with government support under Contract No. 1655740, awarded by the National Science Foundation. The Government has certain rights in the invention.

### BACKGROUND

Enzymes, as efficient biocatalysts in versatile reactions, have been widely explored for use in biotechnological sciences and synthetic applications. Despite significant advantages compared to chemical catalysts, including high specificity and high efficiency, the practical applications of enzymes are often hampered by high cost due to complicated production systems and purification processes, low stability under non-native conditions, and inefficient reusability.

Enzyme immobilization represents a practical way to obtain more stable, active, and reusable biocatalysts. A variety of supporting materials have been explored for enzyme immobilization such as carbon nanotubes (CNTs), metal-organic frameworks (MOFs), synthetic and natural polymers, and hydrogels, as well as zeolites and other inorganic particles. Among them, cellulose, a natural hydrophilic polymer, is one of the most promising supports for enzyme immobilization. Because of its tight crystallite packing resulting from the inter- and intramolecular hydrogen bonds, formation of a cellulose-based supports typically includes dissolution of the natural material by use of certain solvent systems (e.g., NaOH/thiourea/urea, 4-methylmorpholine N-oxide, lithium chloride/dimethylacetamide) followed by precipitation in a swollen form and then derivatization. Unfortunately, this process requires volatile and harmful organic solvents and is energy intensive. Moreover, cellulose supports are often packed into fixed beds that are limited by mass transfer resistance, intolerance of high pressure, inhomogeneous distribution of reactants and products, and poor reproducibility.

A variety of different plant-based materials have been examined for use in forming cellulose-based supports such as edible plants (e.g., apple, carrot, pepper), woody plants (e.g., spruce, balsa) and grasses, among others. Bamboo has also been examined as a desirable raw material for use in forming cellulose-based support materials via the typical formation methods. Compared to the 10-20 years of the life cycle of slower growing timber, bamboo only needs 3-5 years to reach maturity. Moreover, bamboo yields up to 25-fold more material due to fast growth (i.e., up to 100 cm/day), sequesters fourfold more carbon dioxide, and releases 35% more oxygen than woody plants.

What is needed in the art are cellulose-based scaffolds as may be utilized for enzyme immobilization that can exhibit high enzyme loading capability, high activity levels, and reusability. Moreover, methods for forming such scaffolds that can avoid the traditional cellulose extraction techniques would be of great benefit in the art.

### SUMMARY

In one embodiment, disclosed is a cellulose-based scaffold that includes delignified bamboo. The delignified bamboo includes the hierarchical assembly of natural bamboo, retaining the structure of cellulose fibers and hemicellulose chains and defining a plurality of pores having a wide pore size distribution, including large diameter sieve tubes, smaller parenchymal cell lumen, and smaller yet inner cell wall pores. A cellulose-based scaffold as described herein also includes enzymes immobilized at a surface of the delignified bamboo.

Also disclosed is a method for forming the cellulose-based scaffolds. A method can include delignifying a portion of a bamboo culm, oxidizing the surface of the portion to form a plurality of aldehyde groups on the surface, and contacting the surface with an enzyme under suitable reaction conditions to encourage reaction between an amine of the enzyme and an aldehyde group at the surface to form a Schiff base. Following, the Schiff base thus formed can be stabilized by reduction to form an alkylamine linkage between the enzyme and the surface.

Methods for utilizing the cellulose-based scaffolds are also described. For instance, a mixture comprising a substrate can be caused to flow through a bioreactor that retains a scaffold as described. Upon the flow, the substrate can contact an enzyme that is immobilized at a surface of the scaffold under suitable reaction conditions, thereby forming a product that can be collected from the bioreactor. Multi-enzyme systems and methods are also described.

Other features and aspects of the present disclosure are discussed in greater detail below.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 schematically illustrates the microstructure of bamboo and a method for forming a scaffold as described herein.

FIG. 2 provides a schematic illustration of a flow bioreactor including a single enzyme type immobilized on a scaffold as described herein.

FIG. 3 provides a schematic illustration of a flow bioreactor including a dual enzyme reaction scheme.

FIG. 4 schematically illustrates a method for forming a scaffold as described in the examples section herein.

FIG. 5 presents attenuated total reflection-Fourier transform infrared (ATR FTIR) spectra of a bamboo scaffold before and after delignification.

FIG. 6 presents the density and porosity of bamboo scaffolds as described herein before and after delignification.

FIG. 7 presents the pore size distribution curve of a delignified bamboo scaffold obtained by mercury intrusion porosimetry.

FIG. 8 presents a scanning electron microscope (SEM) of a cross-section of raw bamboo.

FIG. 9 presents an SEM of a cross-section of delignified bamboo.

FIG. 10 presents scanning electron microscope (SEM) images of cross sections (1) and parenchymal cells (2) of a delignified bamboo scaffold.

FIG. 11 presents an SEM of a cross-section of a bamboo scaffold following immobilization of an enzyme thereon.

FIG. 12 presents an SEM of a fiber of a bamboo scaffold following immobilization of an enzyme thereon.

FIG. 13 presents the effect of oxidation time (A) and washing (B) on the loading amount of protein (bovine serum albumin; BSA) on a scaffold.

FIG. 14 provides a comparison of immobilized efficiency versus specific surface area for different cellulose based systems.

FIG. 15 graphically illustrates the effect of initial concentration of BSA protein on loading amount on a scaffold with Schiff-base reaction time (A) and washing time (B).

FIG. 16 graphically illustrates the loading amount of a protein on a scaffold before and after washing (A) and the effect of washing on the loading amount of the protein (B).

FIG. 17 illustrates the principle of an enzyme activity test carried out with a scaffold as described herein.

FIG. 18 provides a calibration curve used in the test.

FIG. 19 provides the recovery percentage of product by use of free enzyme.

FIG. 20 provides a comparison of the substrate conversion rate by free enzyme and immobilized enzyme under different conditions.

FIG. 21 provides the recovery percentage of product by use of immobilized enzyme under static conditions.

FIG. 22 provides the recovery percentage of product by use of immobilized enzyme under agitation conditions.

FIG. 23 provides Hanes-Woolf plots of experiments performed with bamboo scaffolds including different amounts of β-glucuronidase (BGU) immobilized thereon and includes the fitting of the Michaelis-Menten equation (insert).

FIG. 24 provides kinetic parameters of an enzyme catalyzed reaction using free enzyme.

FIG. 25 provides the kinetic parameters of the enzyme catalyzed reaction using 5 μg of an enzyme immobilized on a scaffold.

FIG. 26 provides the kinetic parameters of the enzyme catalyzed reaction using 10 μg of an enzyme immobilized on a scaffold.

FIG. 27 provides the kinetic parameters of the enzyme catalyzed reaction using 20 μg of an enzyme immobilized on a scaffold.

FIG. 28 illustrates the remaining activity following multiple cycles and demonstrates the reusability of a scaffold.

FIG. 29 illustrates the storage stability of free BGU compared to a scaffold including BGU immobilized thereon (˜4° C. for 40 days).

FIG. 30 provides recovery percentages from flow bioreactors of different volumes with different flow rates.

FIG. 31 presents the recovery percentage and accumulated 4-nitrophenyl-β-D-glucuronide (pNPG) product from a flow bioreactor as a function of collection volume.

FIG. 32 presents the recovery percentages for flow bioreactors following storage.

FIG. 33 presents the recovery percentage and accumulated oxazepam hydrolyzed product from a flow bioreactor as a function of collection volume at different flow rates.

FIG. 34 presents the recovery percentage and accumulated lorazepam hydrolyzed product from a flow bioreactor as a function of collection volume at different flow rates.

FIG. 35 presents the recovery percentage and accumulated temazepam hydrolyzed product from a flow bioreactor as a function of collection volume at different flow rates.

FIG. 36 presents a schematic diagram and an optical photograph of a flow bioreactor as described herein including tandem enzyme catalysts.

FIG. 37 presents the recovery percentage as a function of collection volume for a glucose substrate fed to the flow bioreactor of FIG. 15.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment, may be used in another embodiment to yield a still further embodiment. Thus, it is intended that the present disclosure cover such modifications and variations as come within the scope of the appended claims and their equivalents.

The present disclosure is generally directed to cellulose-based scaffolds for enzyme immobilization and methods for forming and using the scaffolds. Disclosed scaffolds incorporate delignified bamboo that can be functionalized to include an enzyme immobilized at a surface of the delignified bamboo. Disclosed scaffolds can be utilized in enzyme-based flow bioreactors, successfully providing catalytic reactions as well as subsequent separation in a useful flow-through system. Beneficially, disclosed scaffolds can be formed at relatively low cost and according to environmentally friendly formation methodology, which can allow for recycling of scaffolding materials as well as less use of harmful production materials such as ecologically unfriendly organic solvents. Moreover, the scaffolds can exhibit high compatibility to enzyme structures, allowing for high loading and high activity during use.

Bamboo for use in forming disclosed scaffolds is not particularly limited. A starting material can be of any source bamboo and will include a culm, i.e., a hollow stem, that can be utilized to form a scaffold. The starting material can be pre-treated prior to formation of the scaffold. For instance, the starting material can be washed and pre-treated to remove the epidermal and endodermal layers. Optionally, a pretreatment can also include cutting or chopping the bamboo to a desired size to provide a plurality of individual portions of the culm wall for delignification. Alternatively or additionally, the bamboo can be sized for a particular end use, e.g., smaller sections of the bamboo for loading into a flow bioreactor.

The bamboo walls have a sophisticated structure that includes parenchymal cells as the matrix and vascular bundles as reinforcement, with the vascular bundles being formed of different types and sizes of fibrils including macrofibrils, microfibrils, and elementary fibrils. These hierarchical fibril structures provide a combination of pores of different sizes including relatively large sieve tubes defining conduit pores with a diameter generally of from about 50 μm to about 120 μm, parenchyma cell lumen having a pore size of from about 10 μm to about 50 μm, and inner cell wall pores having a size of from about 0.5 μm to about 10 μm. The porosity and wide pore size distribution as well as the bio-benign characteristics of the delignified cellulose-based material serve to make bamboo an excellent source for a cellulose-based support as it exhibits high surface area and excellent flow characteristics in addition to the environmentally friendly cellulose structure.

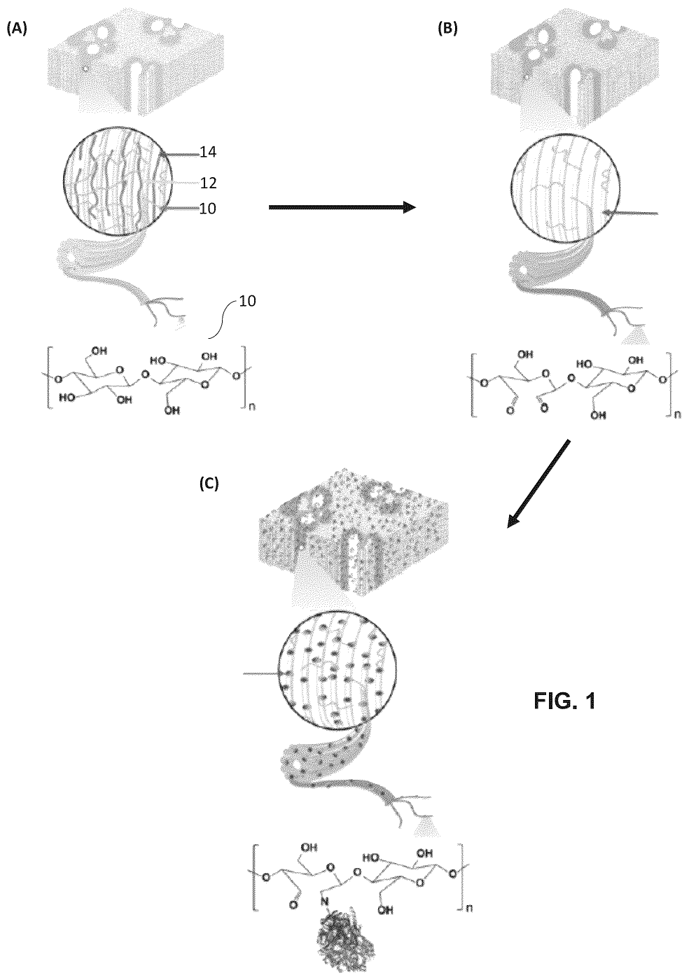

FIG. 1 at (A) illustrates the microstructure of an isolated segment of a typical bamboo culm wall as may be utilized in forming a scaffold. The primary structural component of the fibrils include cellulose 10, hemicellulose 12, and lignin 14. The cellulose 10 is formed of a linear chain of several hundred to many thousands of β-linked D-glucose units as illustrated and generally forms from about 40 wt. % to about 60 wt. % of a typical bamboo wall. The hemicellulose 12 is composed of a number of diverse sugars such as xylose, arabinose, mannose, galactose, and rhamnose, among other possibilities, and generally forms from about 20 wt. % to about 40 wt. % of a typical bamboo wall. As illustrated, the hemicellulose can bind to cellulose (generally in conjunction with pectin) to form the fibrous structural network of the wall. The lignin component 14 generally forms about from about 20 wt. % to about 40 wt. % of the typical bamboo cell wall and is a complex and heterogeneous macromolecule that provides structural and barrier properties to the plant.

The general process for forming a scaffold is schematically illustrated in FIG. 1 and includes delignification of the starting bamboo (A) to form a delignified bamboo (B) followed by functionalization of the delignified bamboo (B) to include enzymes immobilized on the surface of the delignified bamboo (C). As illustrated, the process can retain the overall morphology and cellular structure of the bamboo and through loss of the lignin can increase the internal multiscale porosity, improving enzyme loading level due to the abundance of hydroxyl groups on the extremely large surface area of the porous structure. Moreover, the highly aligned cellulose fibers exhibit excellent liquid transport capacity, leading to a high water flux in the delignified bamboo. As a result the scaffold is chemically amenable for enzyme immobilization due to the abundant reactive groups and hydrophilic chains and the resulting functionalized material is dynamically well-suited for catalysis.

The delignification of the bamboo culm can be carried out according to a facile and cost-effective method that does not require the energy-consuming and environmentally unfriendly cellulose fiber extraction as known in previous delignification methods while retaining the natural structure of the bamboo. The delignification process can also be tuned to control the hydrophobicity of the support surface as well as the polarity of the scaffold environment, which can be important in both successful enzyme immobilization and retention of enzyme activity following immobilization.

The delignification process can include contacting the bamboo with an oxidizing acidic solution under relatively mild processing conditions so as to delignify the bamboo efficiently with little or no damage to the underlying fibrous structure. The oxidizing acidic solution can include an acid and an oxidizing agent that is miscible with the acid. In one embodiment, the acid and the oxidizing agent can both contain only carbon, hydrogen, and oxygen. In some embodiments, at least a portion of the oxidizing acidic solution may be recycled after use.

The acid component of a solution can include an organic acid in the liquid state or in the form of a dissolved solid salt. Examples of suitable acids for use can include, without limitation, carboxylic acids, salts derived from carboxylic acids and acid anhydrides such as the non-phenolic organic acids acetic acid, ascorbic acid, carbonic acid, citric acid, lactic acid, oxalic acid, and propionic acid; and the phenolic organic acids benzoic acid, cafeic acid, chlorogenic acid, ferulic acid, gallic acid, gentisic acid, parahydroxybenzoic acid, vanillic acid, salicylic acid, sinapic acid, syringic acid, resorcinol; as well as any combination thereof.

The oxidizing compound of a solution can include a compound that is capable of capturing one or more electrons. In one embodiment, the oxidizing compound can include a peroxide compound such as hydrogen peroxide or a peroxy acid. A peroxide compound can have the general formula ROOR′, where each of R and R′ is a hydrocarbon chain such as an alkyl, alkyloyl, alkyloxycarbonyl, aryl, aryloyl, or aryloxycarbonyl and mixtures thereof. Examples of hydrocarbon chains are: for an alkyl chain: methyl, ethyl, propyl, butyl, t-butyl, and pentyl, for an alkyloyl chain: ethyloyl, propyoyl, butyloyl and pentoyl; for an alkyloxycarbonyl chain: the carbonate esters such as ethyl, propyl, butyl, pentyl carbonate; for an aryl chain: phenyl, benzyl, chlorobenzyl, naphthyl, thienyl, indolyl; for an aryloyl chain: phenyloyl and naphthyloyl; for an aryloxycarbonyl chain the carbonate esters such as phenyl or naphthyl carbonate.

An oxidizing compound may be combined with an acid so as to produce the oxidizing acidic solution. The acid can exhibit oxidizing properties on its own, but through combination with an oxidizing compound the oxidizing properties can be improved for the delignification process. In some embodiments the combination of the acid and the oxidizing compound may form new reactive entities in the oxidizing acidic solution. Thus, for example, a peroxide may be associated with a carboxylic acid to form a peroxy acid, such as formic peracid or acetic peracid.

In general, the acidic component (i.e., one or more acids) and the oxidizing component (i.e., one or more oxidizing compounds) can be included in an oxidizing acidic solution in a volume:volume ratio of from about 9:1 to about 1:9, such as about 1:1 in some embodiments.

Depending upon the characteristics of the particular compounds used in the oxidizing acidic solution (e.g. pH, dielectric constant, ionic force, acidity, oxidizing or reducing character) the solution can include one or more additional additives. For example, a base, a reducing agent, and/or a salt may be included in an oxidizing acidic solution to modify the solution characteristics.

The bamboo and the oxidizing acidic solution can be held in contact with one another for a period of time, e.g., about 6 hours or more, such as from about 2 hours to about 24 hours, such as about 6 hours. In some embodiments, the mixture can be heated for all or a portion of the contact time, for instance to a temperature of from about 60° C. to about 100° C., such as about 80° C. In one embodiment, the oxidizing acidic solution treatment period can be carried out without mechanical stirring so as to avoid damage to the fibrous structure.

Following this initial treatment, the bamboo can be further contacted with a strong acid solution, e.g., sulfuric acid, nitric acid, or hydrochloric acid, or a combination thereof, for a period of time (e.g., from about 0.5 hours to about 4 hours, such as about 2 hours), optionally while being heated (e.g., to a temperature of from about 80° C. to about 100° C., such as about 95° C.), so as to ensure essentially complete removal of the lignin component of the bamboo. As utilized herein, the term “essentially complete” refers to removal of 99 wt. % or more of the lignin of the starting bamboo material, such as 99.5 wt. % or more, or 99.8 wt. % or more, in some embodiments.

As indicated in FIG. 1 at (B), the delignified bamboo can retain the microstructure of the starting bamboo material, including interconnected porosity having a wide pore size distribution. For instance, pores having an average pore size of greater than about 120 μm can make up from about 5% to about 10% of the total interconnected porosity of the delignified bamboo, pores having an average pore size of from about 50 μm to about 120 μm can make up from about 15% to about 25% of the total interconnected porosity, pores having an average pore size of from about 10 μm to about 50 μm can make up from about 45% to about 55% of the total interconnected porosity, and pores having an average pore size of less than about 10 μm can make up from about 20% to about 30% of the total interconnected porosity.

The delignification process can decrease the density of the bamboo while increasing the overall porosity of the material. For instance, the delignified bamboo (either with or without an enzyme immobilized thereon) can exhibit a density in g/cm³ that is from about 30% to about 40% of that of the starting material. For example, the delignified bamboo can exhibit a density of from about 0.1 g/cm³ to about 0.5 g/cm³, such as from about 0.15 g/cm³ to about 0.4 g/cm³ in some embodiments. Moreover, the total porosity of the delignified bamboo can be more than double that of the starting material. For instance, the total porosity of the delignified bamboo (either with or without an enzyme immobilized thereon) can be from about 75% to about 95%, such as from about 80% to about 90% in some embodiments.

Following the delignification process, the delignified bamboo can be oxidized to form reactive functionality for use in immobilization of an enzyme. More specifically, hydroxyl groups of cellulose at the surface of the delignified material can be oxidized to form dialdehydes as illustrated in FIG. 1 at (B).

An oxidizing agent can be selected that can target the hydroxyl groups of the cellulose to generate a large number of reactive aldehyde groups for high loading capacity while avoiding excessive oxidation of the fibrous structure of the delignified bamboo. By way of example a periodate oxidation compound can be used, such as a sodium periodate in a concentration of about 2M or less, such as from about 0.05M to about 1M in some embodiments. The oxidation can generally take place in the dark, for instance in conjunction with gentle shaking.

Following delignification and surface activation through formation of aldehyde reactive groups, an oxidized bamboo scaffold material can be functionalized with one or more enzymes of choice, as schematically illustrated at (C) of FIG. 1. As is known, the amine groups at the side chain of lysine residues and the N-terminal of proteins are commonly used for enzyme immobilization as the majority of proteins have such amine groups readily available and which are usually exposed and accessible for derivatization without loss of function. As such, an accessible amine group of an enzyme can react with an aldehyde group of the oxidized cellulose surface to form a Schiff base.

To stabilize the Schiff base, the imine linkage can be reduced to form a highly stable alkylamine linkage between the cellulose and the enzyme. To ensure the function of the enzyme, it can be preferred to utilize a relatively mild reducing agent, such as cyanoborohydride, 2-picoline borane, borane diethylamine, etc. Thus, the enzyme can be immobilized on the hierarchical bamboo scaffold with a minimized spacer arm, which can avoid steric hindrance between enzymes and encourage high loading level and high reactivity of the enzyme-functionalized scaffold. For instance, a scaffold as disclosed herein can be functionalized to include immobilized enzyme at a loading level of about 3 mg/g scaffold or greater, such as from about 3 mg/g to about 10 mg/g, from about 3.5 mg/g to about 8 mg/g, or from about 4 mg/g to about 6 mg/g in some embodiments.

When utilizing free enzyme in reaction schemes, aggregation either during the reaction or during storage is a major issue that limits the broader application of enzyme-catalyzed reactions. The highly stable immobilization capability of disclosed scaffolds can prevent enzyme aggregation, providing for sustained activity and reusability as well as excellent storage capability without loss of function. For instance, disclosed scaffolds can maintain about 90% or more of initial enzyme activity levels following 10 reaction cycles or more, such as 12 reaction cycles or more in some embodiments such as from about 10 to about 20 reaction cycles in some embodiments. Moreover, disclosed scaffolds can maintain about 90% or more of initial enzyme activity upon storage at 4° F. or a period of 30 days or more, or more than 40 days in some embodiments.

In one embodiment, scaffolding as disclosed herein can be incorporated in a flow bioreactor that can be designed to include a single enzyme or multiple enzymes, such as in a system designed for a reaction scheme that includes a series of enzyme-catalyzed reactions. Continuous flow biocatalysis by use of a flow bioreactor can be preferred to conventional bulk reaction as the system can provide continuous production as well as facilitated enzyme separation and product isolation.

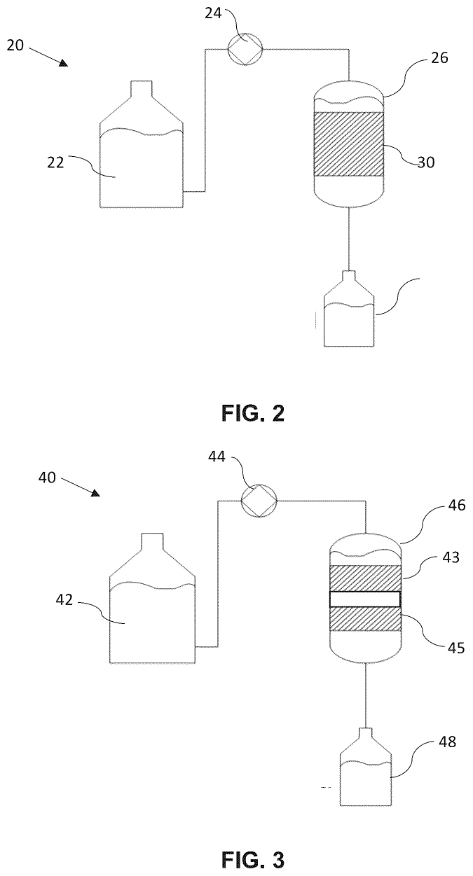

FIG. 2 schematically illustrates one embodiment of a flow bioreactor system 20 that can incorporate a scaffolding material as disclosed herein. The flow bioreactor system 20 can include a substrate source 22, a pump 24, a bioreactor 26, and a product collection 28. The bioreactor 26 can contain the scaffolding 30 that includes the enzyme immobilized therein. The scaffolding 30 can be retained in a bioreactor 26 in portions of any size and arrangement, the particular characteristics of which can be utilized to control flow and pressure characteristics of the system 20. For instance, the scaffolding 30 can be provided in relatively long segments that can be aligned with one another within the bioreactor 26, which may provide for a relatively high flow rate through the bioreactor 26. In other embodiments, the scaffolding 30 can be provided in smaller individual sections that can be packed into the bioreactor in a random, non-aligned fashion, which may provide for a lower flow rate and higher backpressure in the bioreactor 26.

A single enzyme bioreactor system such as that illustrated in FIG. 2 can be designed for any purpose. By way of example and without limitation, in one embodiment disclosed scaffolding materials can be utilized to study drug hydrolysis reactions, e.g., enzyme catalyzed hydrolysis of steroid bioconjugates such as steroid glucuronides, through immobilization of a glucuronidase enzyme such as β-glucuronidase (BGU) on a scaffolding material of a flow bioreactor. BGU has been extensively used in clinical and forensic laboratories and is commonly used to hydrolyze glucuronic acid-conjugated drug metabolites present in different biological fluids. BGU has also been applied to prepare samples for liquid chromatography/mass spectrometry (LC-MS), immunoassay, and other analytical analyses. In previous systems, the enzyme has been utilized as a free enzyme, and as such must be removed from the reaction system after the enzymatic reaction to achieve high sensitivity and accuracy. Such processes are tedious and may bring variations. Disclosed systems can provide great improvement over such previously known processes.

In addition, BGU contains several notable secondary structural forms (e.g., jelly roll barrel and a triose-phosphate isomerase (TIM) barrel). Without wishing to be bound to any particular theory, it is believed that immobilization on disclosed scaffolding materials could improve the higher level structural stability of enzymes like BGU that exhibit multiple structural forms. BGU is also an example of an enzyme that can be reactively tuned by the molecular lipophilicity and polarity and the exterior environment, and such characteristics can be better controlled in disclosed flow-through systems, for instance through modification/control of the carrier fluid, the size and arrangement of the scaffolding retained in the system, etc. Disclosed systems can thus improve separation of reaction products from the enzyme, prevent enzyme leaching, and improve the stability of the tertiary structure of enzymes, as well as improve control of reaction conditions.

Multienzyme systems are also encompassed herein. Multienzyme-based biotransformations have experienced rapid growth for both scientific and industrial applications in recent decades. Conventionally, multienzymatic transformation is carried out in a one-pot reaction system. This approach has limitations due to potential cofactor competition, product mutual inhibition, etc. Moreover, the separation of the multistep enzymatic reaction has disadvantages including high operation costs, instability of intermediate products, and low yields. Disclosed multienzyme systems can be used in one embodiment to catalyze tandem reactions under a continuous flow setup for synthetic and other applications.

FIG. 3 provides an illustration of one embodiment of a multienzyme flow bioreactor 40 that can incorporate multiple enzymes in a tandem reaction system. As illustrated, the bioreactor 40 can include a substrate source 42, a pump 44, a bioreactor 46 and a product collection 48. The bioreactor 46 can retain a first enzyme on a first scaffolding 43 and a second, different enzyme on a second scaffolding 45, with the first and second scaffolding 43, 45 arranged such that the flow carrying the substrate will contact each scaffolding in succession. Of course, a multienzyme system can include more than two enzymes. Moreover, each scaffolding section can include multiple different enzymes in some embodiments. Additionally, there is no requirement that a multienzyme system includes the different enzymes in a single bioreactor, and a system can include multiple separate and distinct bioreactors in fluid communication with one another.

The cellulose-based scaffolds disclosed herein can provide economical and bio-safe systems for immobilization of enzymes without destruction of enzyme structure or activity with excellent storage and reusability capabilities. Moreover, disclosed scaffolds can be used in multiple applications including, without limitation, biomanufacturing, catalysis, bioanalysis, and separations, as well as other enzymatic applications.

The present disclosure may be better understood with reference to the following example.

### Example

Moso bamboo was collected from the Wuyishan bamboo forest in China. One- to two-year-old bamboo culms were selected. The epidermal and endodermal layers were removed, and the culms were immersed in hydrogen peroxide (H₂O₂, 30%) at 70° C. overnight. Samples were cut to dimensions of 5 mm×6.5 mm×25 mm (longitudinal×radial×tangential), and the pieces were stored at 20° C./65% relative humidity before treatment.

β-glucuronidase (BGU) (50 kU/mL, 1.0 mg/mL), which was highly purified, filter sterilized, and genetically modified, was provided by Integrated Micro-Chromatography Systems, Inc. Horseradish peroxidase (HRP) was purchased from VWR. 2,2′-Azino-bis(3-ethylbenzothiazoline-6-sulfonic acid) diammonium salt (ABTS, C₁₈H₁₈N₄O₆S₄·(NH₃)₂) and 4-nitrophenyl-β-D-glucuronide (pNPG, C₁₂H₁₃NO₉) were purchased from Chem-Impex. Glucose oxidase (GOx), bovine serum albumin (BSA), i-propanol (C₃H₈O), glycine (C₂H₅NO₂), and sodium hydroxide (NaOH) were purchased from Sigma. Sulfuric acid (H₂SO₄, 98%), glacial acetic acid (HAc), D-glucose, hydroxylamine hydrochloride (NH₂OH·HCl), sodium cyanoborohydride (NaCNBH₃), and fluorescein-5-maleimide were purchased from Fisher Scientific. Hydrogen peroxide (H₂O₂, 30 wt. %) was purchased from Macron Fine Chemicals. 4-Nitrophenol (pNP) was purchased from Aldrich. Milli-Q water deionized using a Synergy Ultraviolet (UV) Water Purification System was used. All chemicals and solvents used were of analytical grade. Unless otherwise noted, 0.1 M potassium phosphate, pH 7.4, was used as a buffer.

### Delignification Procedure

The moso bamboo pieces were immersed in a beaker with an equal-volume mixture of H₂O₂ solution and HAc for 6 h at room temperature. The mixture was heated at 80° C. until the color of the pieces changed from yellow to white. The white pieces were then immersed in H₂SO₄ aqueous solution (3 wt. %) at 96° C. for 2 h. The delignified bamboo scaffolds thus formed were washed thoroughly with water before use. The samples were stored in water and cut into cylinder shapes with a diameter of 5 mm and a height of 5 mm (22 mg±2 mg). Each delignified bamboo scaffold was placed on the bottom of a plastic column during use.

### Characterization of Delignified Bamboo

Brunauer-Emmett-Teller (BET) measurements were carried out on a Micromeritics TriStar II 3020 instrument through a N₂ physisorption surface at a relative pressure between 0.01 and 0.99. The bamboo and delignified bamboo scaffold samples were dried in an oven at 100° C.; then, ATR-FTIR spectroscopy was performed at room temperature across the frequency range of 4000-650 cm⁻¹ on a spectrum system (IS 50, Thermo Nicolet Ltd.), with 64 FTIR scans per spectrum. The surface morphology was observed using a Tescan Vega 3 SBU variable-pressure scanning electron microscopy (SEM) system after gold sputtering (240 seconds to give an ˜40 nm gold conductive layer; Denton Desk II sputter coater). The porosity was determined using the gravimetric method. Specifically, a piece of scaffold was lyophilized (Labconco Freeze Dryers FreeZone 4.5 Liter) at −80° C. with 0.003 MPa vacuum overnight and subsequently weighed using an analytical balance (Denver Instrument PI series). The sample was fully immersed in i-propanol for 1 day for complete alcohol infiltration. Then, the sample was cleaned to remove residue from the surface and weighed. The porosity (n) was estimated using eq. 1:

$$n = \frac{{V}_{pore}}{{V}_{total}} = \frac{{m}_{IPA} / {ρ}_{IPA}}{{V}_{total}} \qquad ( 1 )$$

in which
   V is volume
   m is mass
   p is density, and
   IPA indicates i-propanol.
Three independent measurements were performed on each sample.

The pore structure of the delignified bamboo scaffold after lyophilization was evaluated by mercury penetration using an Autopore IV 9500 mercury intrusion porosimeter (Micromeritics) with the contact angle employed as 130° and measuring range as 0.005-800 μm.

### Preparation of the Oxidized Bamboo Scaffold (OBS)

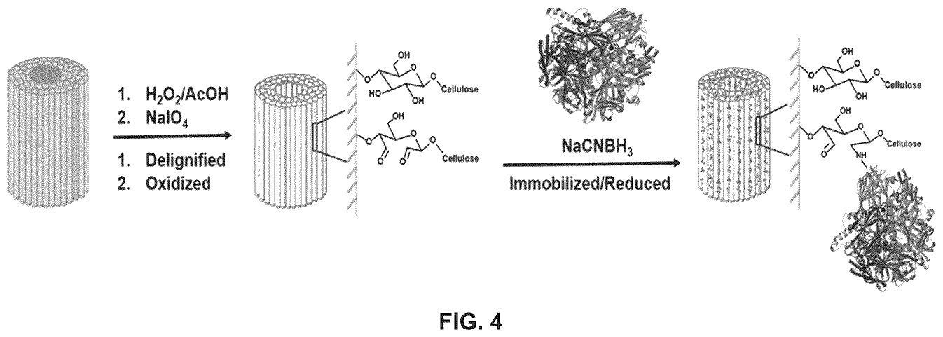

The processing route utilized from native bamboo to protein-immobilized bamboo in this example is depicted schematically in FIG. 4. As described in more detail below, the delignified bamboo scaffold was fabricated in a green route using H₂O₂-Hac. This mild process employing peracetic acid (PAA) generated from an HAC and H₂O₂ mixture can delignify bamboo efficiently with little fiber damage. In the process, the bamboo bulk was treated with H₂O₂-HAc (V:V=1:1) solution at 80° C. Mechanical stirring was avoided. The reagents used in the method only contain C, H, and O and do not introduce additional spacer atoms, which is helpful for the subsequent cellulose oxidation reaction. The hydroxyl group at C2 and C3 of the unit of surface cellulose was oxidized by NaIO₄ to form dialdehyde, which could react with the amino group (—NH₂) on the protein via the Schiff base reaction and be reduced into an alkylamine linkage subsequently in one pot by NaCNBH₃.

The oxidized bamboo scaffold was prepared by the sodium periodate oxidation method. First, the bamboo scaffold was washed with 100 mL of Milli-Q water at 20-25 mL/min and then immersed in 500 μL of sodium periodate (0.2 M) for 2 h. The immersion was performed in the dark at room temperature with gentle shaking. After oxidation, the excess IO₄⁻ and Na⁺ were removed by washing with approximately 600 mL of flowing Milli-Q water with 20-25 mL/min.

Following formation the OBS was lyophilized to test the aldehyde content by deduction from the quantitative reaction with NH₂OH·HCl. In a typical experiment, two pieces of OBS were reacted with 20 mL of 0.25 M NH₂OH·HCl solution for 3 h at room temperature. The pH of the hydroxylamine solution alone and that with OBS were initially 3.2. After 3 h, the pH of the mixture decreased due to HCl release. The solution was then titrated with 0.01 M NaOH solution back to pH 3.2, and the content of aldehyde (CHO) was calculated by the following eq. 2:

$$content of C H O ( μmol / mg ) = \frac{{V}_{eq} \times {C}_{NaOH}}{m} \qquad ( 2 )$$

in which
   V_{eq} is the volume (mL) of the standard NaOH solution added to reach pH 3.2,
   C_{NaOH} is the concentration (mol/L) of the standard NaOH solution, and m (mg) is the weight of OBS.

### Immobilization of BSA and BGU

To determine the immobilized protein capacity of the OBS, BSA labeled with fluorescein-5-maleimide (F-BSA) was prepared. BSA was reacted with fluorescein-5-maleimide overnight at 4° C. at a ratio of 1:2 in the dark followed by removal of nonreacted fluorescein by dialysis using a dialysis tube with 10 kDa cutoff. During the dialysis procedure, dialysis buffer of least 300 times the volume of the sample was used; the dialysis buffer was changed every 6 h for a total of three times, and the UV absorbance of the solution at 498 nm before and after labeling was detected by spectrophotometry (Nanodrop 2000c, Thermo Scientific). F-BSA was protected from light and stored at 4° C. The amount of immobilized protein was measured by detecting the difference between the initial and final concentrations of F-BSA using fluorescence spectrophotometry at 518 nm with an excitation wavelength of 494 nm before and after the Schiff base immobilization reaction (Varian Cary Eclipse, Agilent). The F-BSA solution was tested as a negative control at the same time. F-BSA at 0.1, 0.4, and 1.0 mg/mL concentration was used. Each scaffold was washed in 5 mL×1.5 mL of phosphate buffer (0.1 M, pH 7.4) following the immobilization reaction to remove nonspecifically bound proteins, during which the buffer was changed at 1, 3, 8, 24, and 54 h. The loading amount of the immobilized enzyme was calculated by eq. 3:

$$loading amount ( mg / g ) = \frac{( {W}_{0} - {W}_{t} )}{m} \qquad ( 3 )$$

in which
   W₀ is the loading amount of enzyme before washing (i.e., after the Schiff base reaction for 16 h),
   Wₜ is the accumulated amount of enzyme in the washing solution, and
   m is the weight of the scaffold.

For BGU immobilization, the OBS samples were immersed in the BGU solution (1.0 mg/mL, 500 UL in 0.1 M, phosphate buffer, pH 7.4); 3 mg of NaCNBH₃ was added, and the mixture was reacted at 4° C. for overnight to maximize the protein loading. Following, the scaffold was immersed in 1.5 mL of 1.0 mol/L glycine solution for 6 h and then immersed in 5 mL×1.5 mL of phosphate buffer to remove nonspecifically bound BGU. The loading amount of BGU was calculated by measuring UV absorbance at 218 nm before and after washing.

The theoretical maximum loading capacity was related to the specific surface area (S) of the delignified bamboo scaffold, which was 0.45 m²/g. The theoretical maximum specific adsorption capacity was calculated by eq. 4 with an assumption that the enzyme is a spherical nanoparticle with a radius of 2 nm:

$$theoretical loading capacity ( mg / g ) = \frac{S \times {M}_{enzyme}}{π {r}^{2} {N}_{A}} \qquad ( 4 )$$

in which
   S is the specific surface area of the OBS,
   M_{enzyme} is the molecular weight of the enzyme (67 kDa for the model protein F-BSA, 80 kDa for the BGU in the monomer form),
   r is the radius of the enzyme (2 nm), and
   N_{A} is Avogadro's number (6.02×10²³).

### Measurement of BGU Activity and Kinetic Study

BGU is stable for up to 3 h at incubation temperatures of up to 60° C. in the range of pH 4 to pH 10. However, when using different percentages of organic solvents, a correction factor was applied to the enzyme activities. BGU showed different catalytic activities toward different glucuronidated substrates. Herein, pNPG was used as a representative substrate for enzyme activity evaluation. The concentration of the pNP product was determined by monitoring the absorbance at 400 nm as previously described. In brief, 500 μL of pNPG (2.0 mM, dissolved in 0.1 M phosphate buffer, pH 7.4) was added to a device including immobilized BGU on OBS (BGU@OBS) and incubated at room temperature under static and shaking conditions. The product pNP solution was tested in a nanodrop at different reaction times. To test the reusability and storage stability of BGU@OBS, the BGU@OBS was washed after the reaction with 100 ml of water and prepared for the next cycle. Three independent replicates were performed under the same conditions. The Michaelis constant, Kₘ, and vₘₐₓ of free and BGU@OBS were determined using a plate reader. The substrate concentration was set from 0.01 to 2.0 mM. To obtain BGU@OBS samples carrying different amounts of immobilized BGU (i.e., 20, 10, and 5 μg), BGU@OBS blocks were cut into 4, 8, and 16 splits in the growth direction.

### Flowing Biocatalysis

To examine flowing systems, scaffolds carrying immobilized BGU were placed in a sample holder, and the flow rate of samples with different nNPG concentrations (4 μM, 80 μM, 2.0 mM in 0.1 M phosphate buffer, pH 7.4) was held constant at each rate (5, 25, 25, 50 μL/min) and controlled using a Legato 180 syringe pump (KD Scientific). The flow-through was collected and then tested for UV absorbance at 400 nm. The activity of BGU@OBS was tested immediately and after storage for 2-49 days.

To test the hydrolysis of glucuronidated drugs of abuse in synthetic urine (Surine), three kinds of benzodiazepine drugs (oxazepam, lorazepam, temazepam) were used. A spiked Surine mixture (glucuronides: 23.8 ppb, internal standard: 4.76 ppb) was freshly prepared, and the flow-through reaction was executed by mounting one piece (diameter: 5.5 mm, height: 5 mm) of BGU@OBS. The flow rate was controlled so as to remain constant at the selected rate (5, 10, 50 μL/min). 250 μL fractions of the sample were collected, quenched using 160 μL of elution solvent (5% FA in MeOH), transferred to a b-Gone Plus Plate, centrifuged at 500 g for 1 min, transferred at a volume of 200 μL into separate wells, and diluted into 600 μL. The collected samples were analyzed by liquid chromatography-mass spectrometry (LC-MS). Four controls were characterized in parallel: (1) blank scaffold under static conditions, (2) BGU@OBS under static conditions, (3) free BGU, and (4) unhydrolyzed sample (Surine sample only).

A flow reaction was carried out with immobilized HRP and GOx in which reactor was prepared by mounting two pieces of OBS scaffold carrying immobilized GOx (GOx@OBS) and two pieces of OBS scaffold carrying immobilized HRP (HRP@OBS). The substrate solution came into contact with immobilized GOx first and thereafter was transported to the immobilized HRP. The flow rate was set as 10 μL/min, and the UV absorbance of the ABTS⁺ product was measured at 415 nm. The substrate contains D-glucose (50 mM) and ABTS (2 mM) in phosphate buffer (0.1 M, pH 7.4).

### Delignification and Characterization

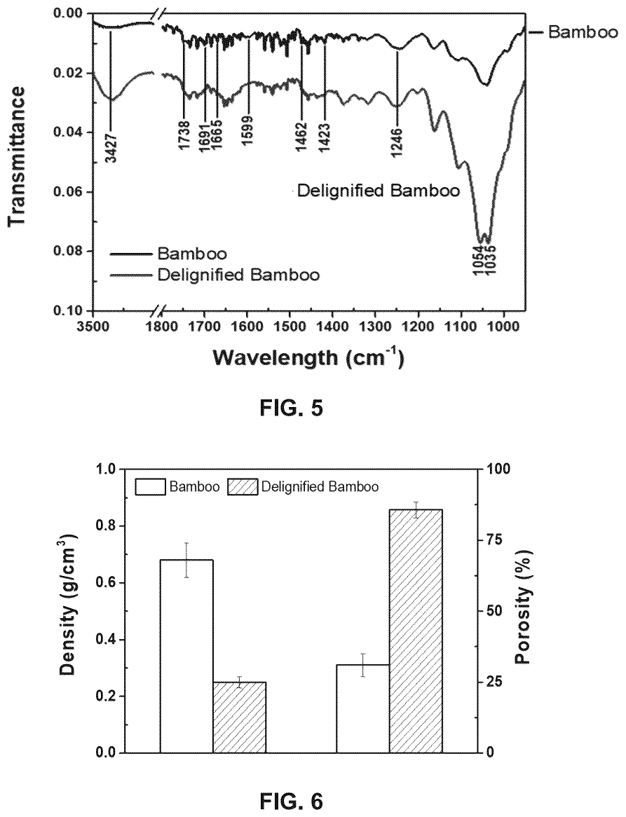

After delignification, the bamboo color turned from slight yellow to white. The ATR-FTIR spectral analysis (FIG. 5) indicated an effective delignification process. The specific split peaks at 1054 and 1035 cm⁻¹ were attributed to cellulose-I and cellulose-II, respectively. The removal of lignin was confirmed by the absence of bands at 1599, 1423, and 1462 cm⁻¹, which were assigned to aromatic skeletal vibrations and C—H deformation combined with aromatic ring vibrations. The obvious reduction in the intensity of peaks at 1691 and 1655 cm⁻¹ was related to carbonyl stretching vibrations in lignin. Partial hemicellulose removal was found based on the disappearance of the peak at 1738 cm⁻¹ corresponding to the carbonyl stretching vibration of hemicellulose, and the small change of the absorption peak at approximately 1246 cm⁻¹ was due to the C—O linkage in xylan. The predominant absorption at 3427 cm⁻¹ was assigned to —OH stretching because the cellulose portion was exposed significantly after the removal of lignin and hemicellulose, which could lead to increasing interactions via hydrogen bonding.

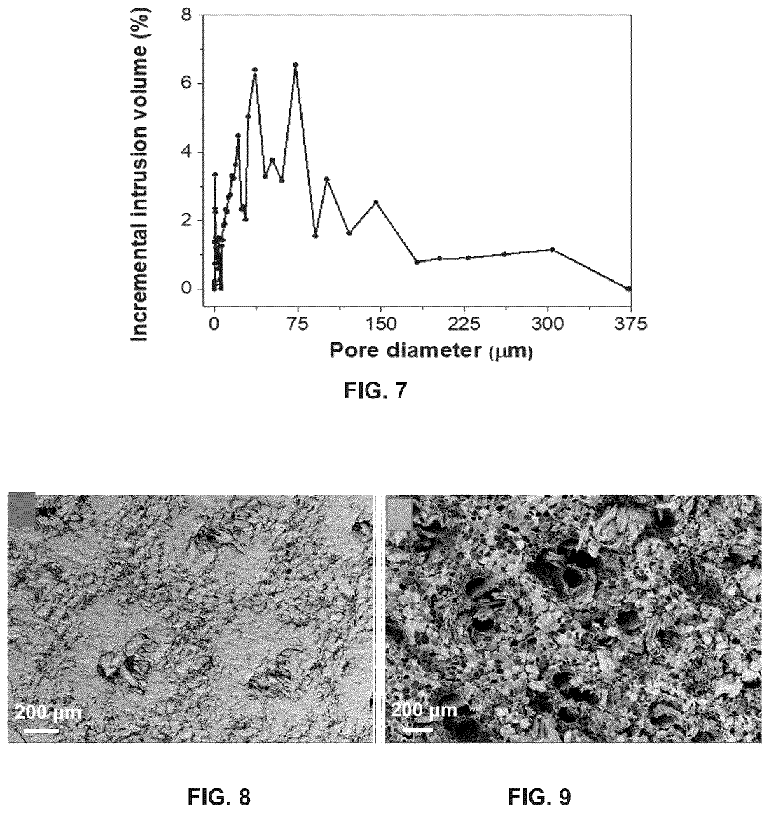

After delignification, the density of bamboo decreased from 0.68±0.06 g/cm³ to 0.25±0.01 g/cm³ (FIG. 6). The loss of hemicellulose and lignin contributed to a mass loss of 63% after the process. Through utilization of eq. 1, it was determined that the porosity of the scaffold increased from 31±4 to 86±3% upon delignificaiton. The pore size distribution results (FIG. 7 and Table 1) showed that the hierarchical delignified bamboo included vessels and sieve tubes having the largest conduit pores with a size of 50-120 μm diameter (20%), parenchyma cell lumen with a medium size of 10-50 μm (46%), and the smallest pores in the inner cell wall with a size range of 0.5-10 μm (24%), which is consistent with reported results.

TABLE 1

| | |
|---|---|
| Total connected porosity (%) | 80.8 |

| | | | |
|---|---|---|---|
| Pore size | <0.05 | μm | 0 |
| distribution (%) | 0.05-0.50 | μm | 2.8 |
| | 0.50-10 | μm | 23.8 |
| | 10-50 | μm | 46.2 |
| | 50-120 | μm | 19.9 |
| | 120-375 | μm | 7.3 |

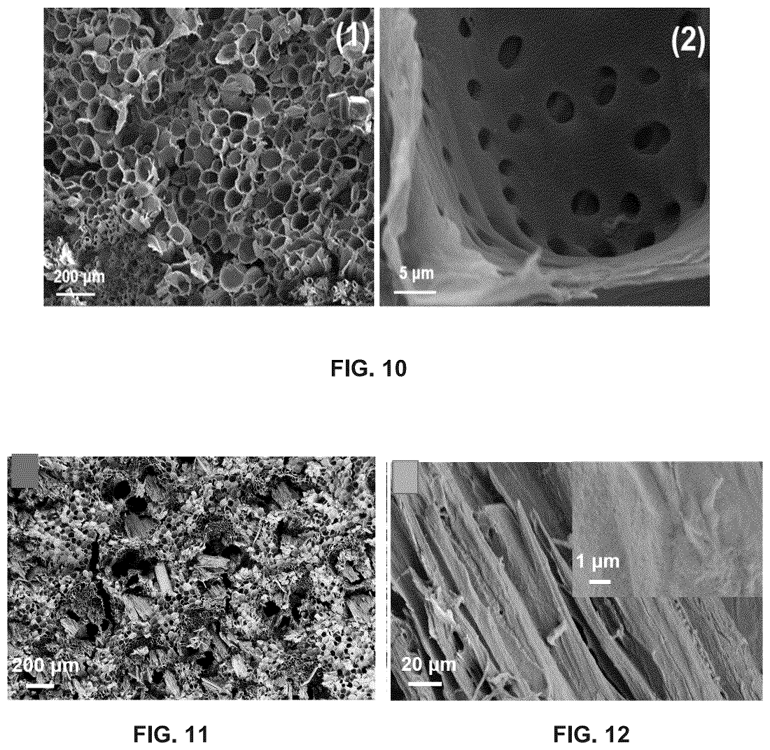

SEM images of raw bamboo and the delignified bamboo scaffold (FIG. 8, FIG. 9) show that the micropores were exposed significantly after delignification. SEM analysis (FIG. 10 at (1), FIG. 11, FIG. 12) showed vast micropores in the range of 15-120 μm with a fractured surface. The higher porosity would typically lead to a higher hydraulic conductivity, which will be helpful for diffusion and separation during the chemical process. A large number of pits ranging from 0.5 to 10 μm in diameter were sustained in the parenchymal cells (FIG. 10 at (2)), which were used for nutrient transport and cell attachment in the live organism. These pits provide short diffusion distance while offering sufficient space due to the anisotropic and hierarchical structural feature, which is beneficial for enzyme immobilization and biocatalytic reactions.

### Oxidation of the Bamboo Scaffold for Enzyme Immobilization

The delignified bamboo scaffold was oxidized using NaIO₄ to give the oxidized bamboo scaffold (OBS), which was then used for protein immobilization. The content of CHO (as determined according to eq. 2) was correlated to the reaction time. Around 0.61 μmol/mg CHO of OBS was observed following 3 h of treatment (Table 2, below), which is ˜2-fold the result found in the literature (0.32 μmol/mg).

TABLE 2

| | | Calculated | Immobilized |
|---|---|---|---|
| Oxidation | Content of CHO | Loading Capacity | Loading Capacity^{[a]} |
| time (h) | (μmol/mg) | (mg/g) | (mg/g) |
| 1 | 0.31 ± 0.05 | 3066 ± 216 | 3.7 ± 0.1 |
| 2 | 0.56 ± 0.03 | 4166 ± 218 | 3.6 ± 0.3 |
| 3 | 0.61 ± 0.04 | 6153 ± 317 | 3.6 ± 0.6 |

^{[a]}Data are the means ± standard deviation of three replicates.

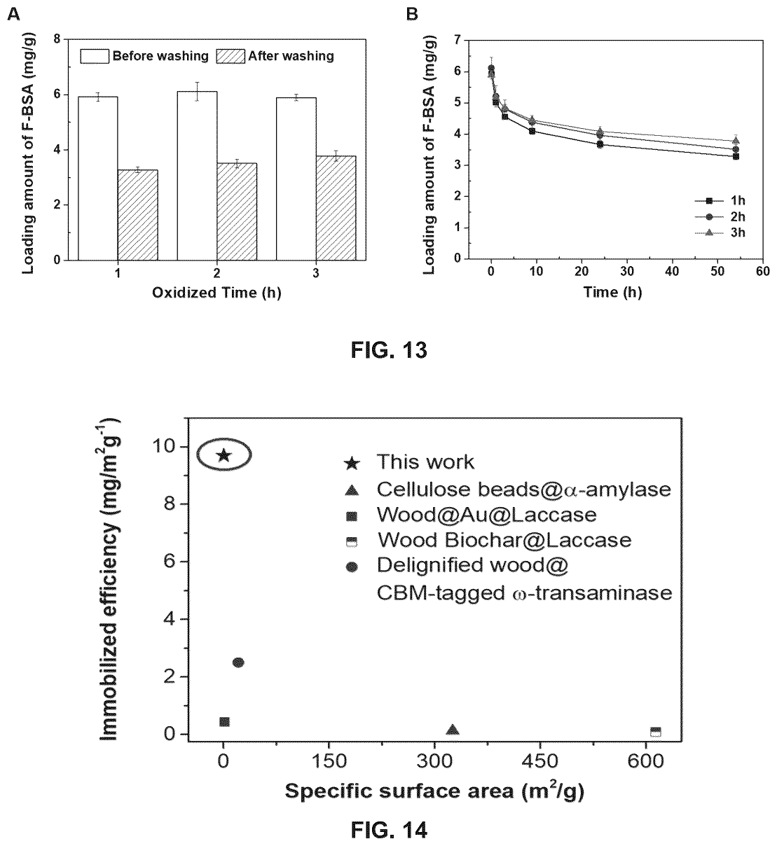

To quantify the efficiency of immobilization, fluorescein-5-maleimide-labeled BSA (F-BSA) was used as a model protein. As shown in Table 2, the loading amount of F-BSA per gram of OBS was 3.6-3.7 mg (as determined by eq. 3). The loading amounts of F-BSA on different OBSs with varied contents of CHO were found similar (Table 2, FIG. 13), which almost reached the highest loading capacity (3.98 mg/g as the theoretical loading capacity, which was estimated based on the inner specific surface area of the OBS and the dimension of the BSA particle, eq. 4). This result supports the expected high efficiency of the immobilization method for delignified bamboo.

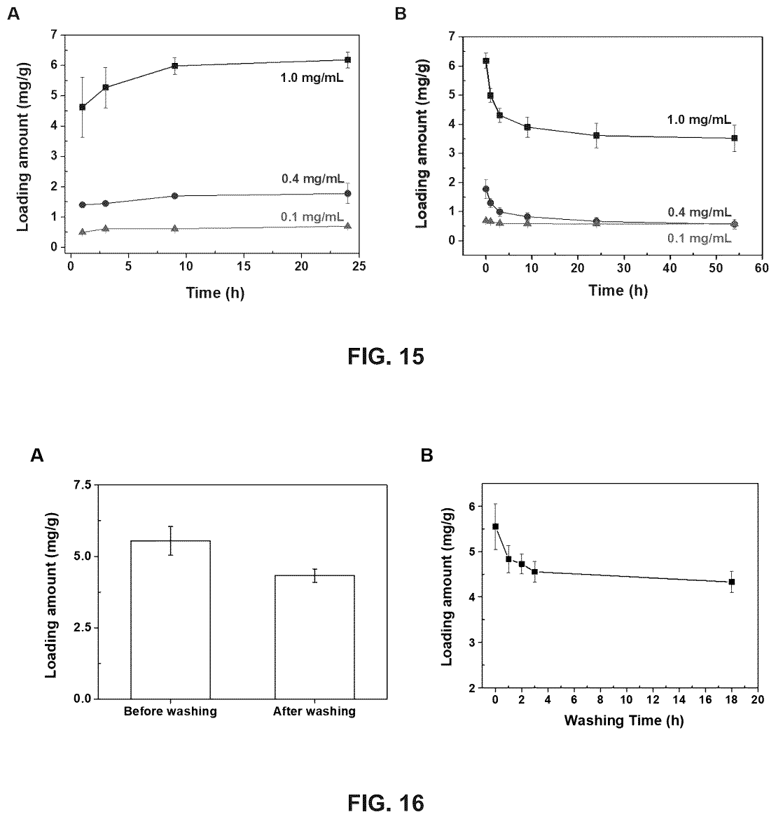

As shown in FIG. 14 and Table 4, below, the protein loading efficiency of the OBS (8.09 mg/m²·g⁻¹) was 3-20 times that of delignified wood and other cellulose-based supports with comparable or much bigger specific surface area. Furthermore, when a high concentration of F-BSA (i.e., 1.0 mg/mL) was used, the protein loading was ˜6-fold that at low concentrations (0.1-0.4 mg/mL, Table 3; FIG. 15), indicating the diffusion rate as an important factor affecting the immobilization process. Similarly, when 1.0 mg/mL BGU was used for immobilization, 4.4±0.2 mg/g loading capacity was identified (FIG. 16, Table 3), which was close to the calculated theoretical capacity (4.8 mg/g, Table 3).

TABLE 3

| | Concentration of | Theoretical loading | Immobilized Loading |
|---|---|---|---|
| Enzyme | enzyme (mg/mL) | Capacity (mg/g) | Capacity^{[a]} (mg/g) |
| F-BSA | 0.1 | | 0.66 ± 0.09 |
| F-BSA | 0.4 | 3.98 | 0.61 ± 0.20 |
| F-BSA | 1.0 | | 3.64 ± 0.26 |
| BGU | 1.0 | 4.76 | 4.36 ± 0.23 |

^{[a]}Data are the means ± standard deviation of three replicates

TABLE 4

| | | | Specific | | |
|---|---|---|---|---|---|
| | | | surface | Immobilized | Immobilized |
| | Protein/MW | Immobilized | area | amount | efficiency |
| Saffold | (kDa) | method | (m²/g) | (mg/g) | (mg/m² · g⁻¹) |
| Wood@Au | Laccase (97) | Au adsorption¹ | 1.3 | 0.6 | 0.43 |
| Delignified | CBM-tagged | CBM-cellulose | 21 | 52.4 | 2.50 |
| wood | ω-transaminase | affinity binding | | | |
| | (49) | | | | |
| Cellulose | α-amylase (58) | Covalent | 325.3 | 41.2 | 0.12 |
| beads | | binding of | | | |
| | | glutaraldehyde | | | |
| Wood | Laccase (97) | Biochar | 613.6 | 11.1 | 0.02 |
| biocar | | adsorption | | | |
| BGU@OBS | BSA (67) | Covalent | 0.45 | 3.8 | 8.09 |
| | | binding of | | | |
| | | Schiff-base | | | |

### Activity of the BGU@OBS and Kinetic Analysis

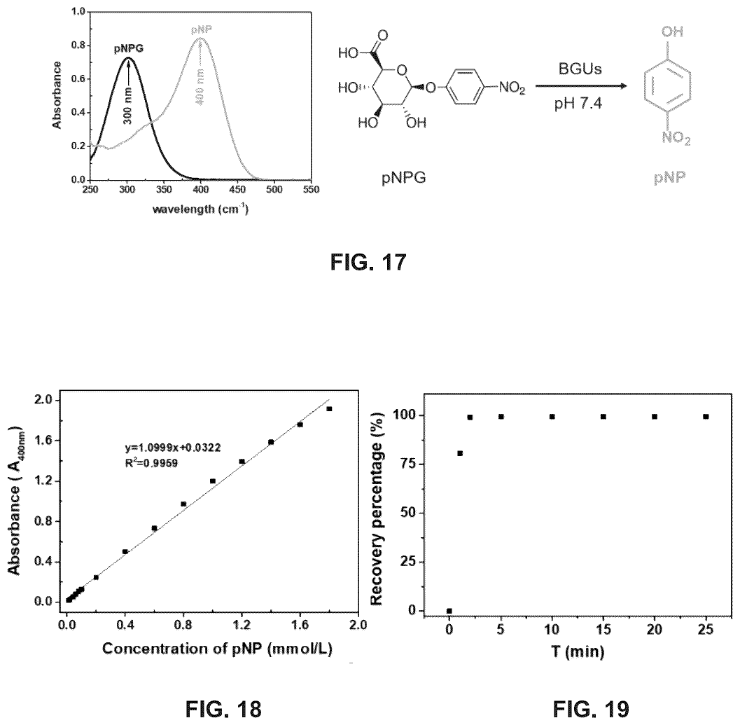

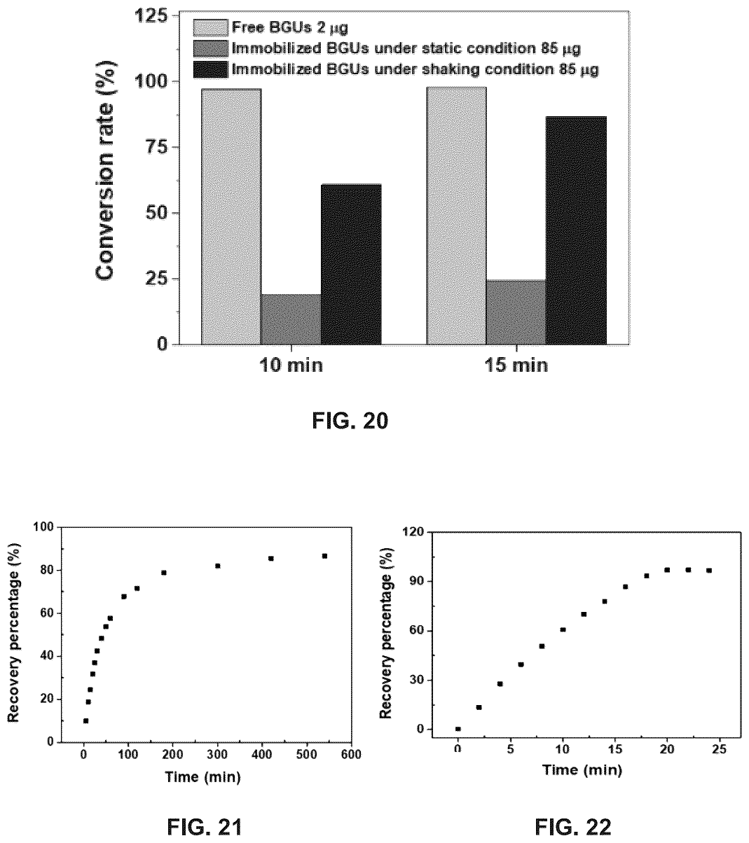

To evaluate the activity of BGU@OBS, a chromogenic reaction with 4-nitrophenyl-3-D-glucuronide (pNPG) as the substrate was employed (FIG. 17, FIG. 18 providing the calibration curve). The reactions were carried out in phosphate buffer (pH 7.4) at room temperature, which is a common condition for BGU-based catalysis. As shown in FIG. 19-FIG. 22, during the 15 min reaction, more than 86.8% conversion rate was achieved by BGU@OBS under shaking, while much lower conversion (24.5%) was observed without shaking, implying that the enzyme's function was largely sustained by comparing the conversion rate of the commercial enzyme.

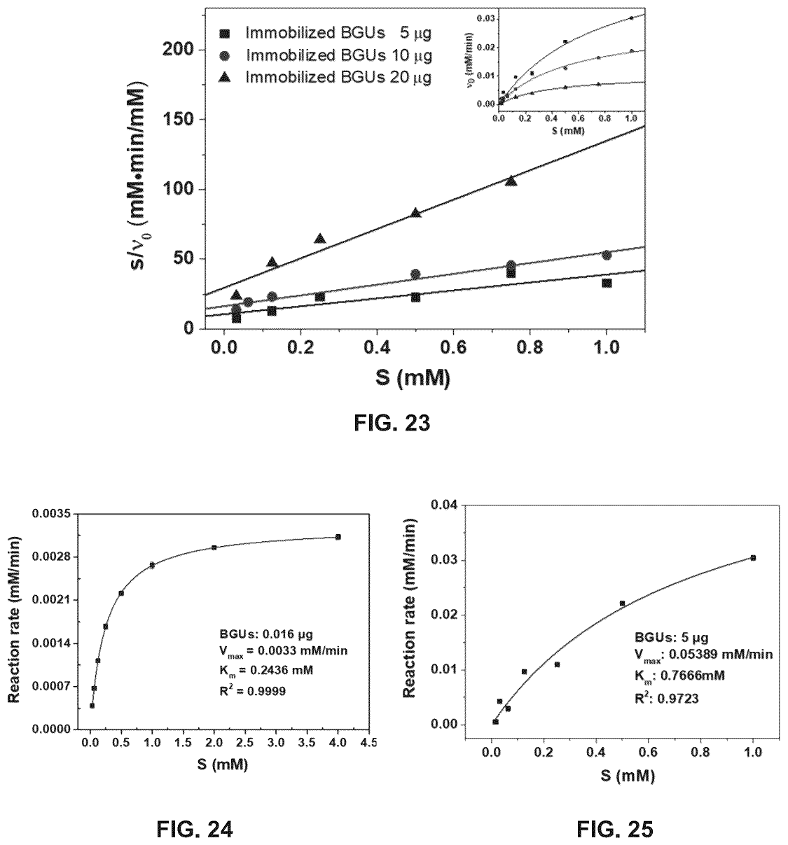

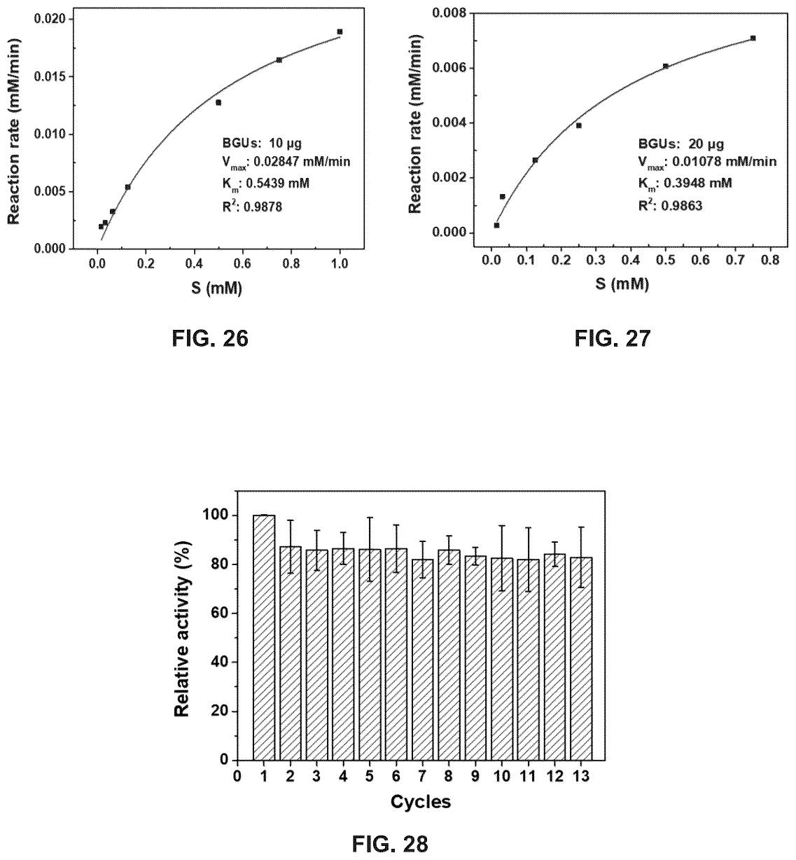

To further characterize the reaction kinetics, the initial reaction rate of BGU@OBS was assessed with increasing concentration of pNPG from 0.01 to 2.0 mM. The reactions with free enzyme (0.016 μg) were carried out with the same conditions for comparison. Different sizes of BGU@OBS with 5, 10, and 20 μg of enzyme were used in the assay for comparison. The Hanes-Woolf plotting analysis of pNPG hydrolysis showed a single straight line (FIG. 23), which supports a Michaelis-Menten behavior of BGU@OBS (FIG. 23, insert). As shown in Table 5, below, and FIG. 24-FIG. 27, BGU@OBS showed a slower reaction kinetic behavior compared to that of the free enzyme in solution. BGU@OBS had a ˜3× higher apparent enzyme-substrate affinity (Kₘ) and a ˜19× lower catalytic activity (K_{cat}) than those of the free enzyme in solution.

TABLE 5

| | C_{BGU} | Kₘ^{b} | νₘₐₓ^{c} | K_{cat}^{d} |
|---|---|---|---|---|
| BGU | (nmol/L) | (nmol/L) | (μmol/L min) | (S⁻¹) |
| Free BGU | 0.24 | 0.24 | 3.30 | 229.28 |
| 0.016 μg | | | | |
| BGU@OBS | 74.96 | 0.77 | 53.89 | 11.98 |
| 5 μg | | | | |
| BGU@OBS | 149.93 | 0.54 | 28.47 | 3.16 |
| 10 μg | | | | |
| BGU@OBS | 299.85 | 0.39 | 10.78 | 0.60 |
| 20 μg | | | | |

The reaction rate was determine with 5 min reactions. In the assay, 0.016 mg of free BGU and 5-20 mg of immobilized BGU were used.
^{b}Kₘ = Michaelis-Menten constant in mM.
^{c}νₘₐₓ = maximum specific activity in mM/min.
^{d}The turnover rate K_{cat} = □ₘₐₓ/E (enzyme concentration.

The results obtained for the free enzyme were comparable with published data. Generally, the biocatalytic processes involve both molecular and convective diffusion including substrate diffusion to catalytic sites and product diffusion back to the solution. Moreover, the density of BGU immobilized at different locations of the hierarchical scaffold should be different due to the gradient distribution of the vascular bundles from the inner skin to the outer skin. In the system herein, molecular diffusion could be affected by the bamboo structure including their internal geometry. The rates of adsorption and desorption of small molecules (both the substrate and the product) by bamboo are related to the external diffusion coefficient and the internal resistance (due to the boundary layer, the diffusion in the materials, etc.). Under some conditions, the apparent diffusion coefficient could be positively correlated with the materials' thickness. All these factors may cause a slightly decreased Kₘ value with a larger size of enzyme@OBS. Similarly, with plenty of micron-level pore sizes, the diffusion of enzymes could be dramatically affected by capillarity. The slower reaction rate displayed in the larger BGU@OBS sample further implied the effect of diffusional resistances on the enzymatic reactions, which has been described in heterogeneous enzyme systems. Without wishing to be bound by theory, other reasons that are believed to have caused the activity decrease may include (1) less conformational flexibility of enzymes because of immobilization; (2) structural changes occurring during enzyme immobilization; 85 and (3) an unmixed solvent layer around the support surface, which is product-rich and substrate-depleted, hence leading to slower reaction kinetics.

### Reusability and Stability of the BGU@OBS

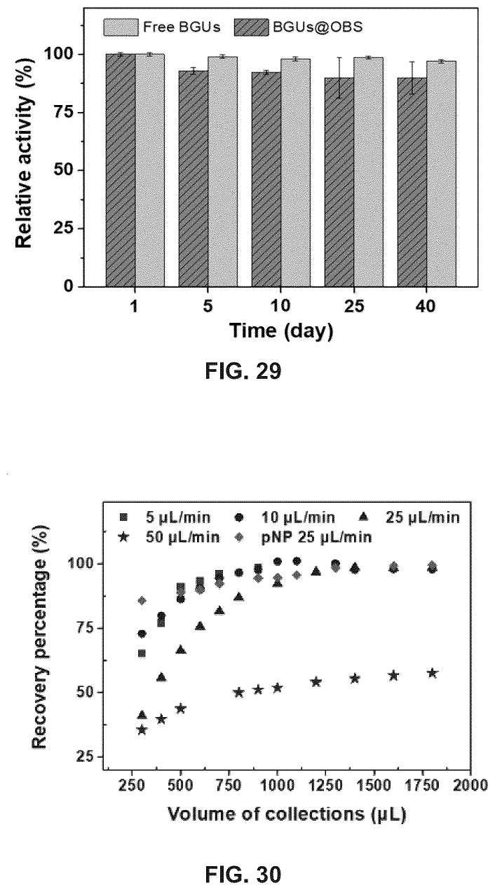

The same batch of BGU was used for enzyme immobilization and characterization to avoid batch-to-batch variations. As shown in FIG. 28 and FIG. 29, the BGU@OBS displayed excellent reusability and stability. The relative activity of immobilized BGU in the first run was set as 100%. After 13 cycles, ˜90% of enzyme activity was maintained (FIG. 28). FIG. 29 shows that ˜90% of enzyme activity of the immobilized BGU was maintained after more than 40 days of storage at 4° C. The slight activity decrease could be attributed to the leakage of BGU from the scaffold due to the degradation through ring opening during oxidation, which may disrupt the ordered structure of cellulose of the scaffold. The error bars represent the standard deviation of the mean of triplicate samples.

Flowing Reaction with a Single Enzyme and Bioseparation.

A continuous flow reactor using BGU@OBS was constructed as shown in FIG. 2. As shown in FIG. 30, the conversion rate increased with decreasing flow rate. The optimal flow rate that obtained >99% conversion was found as 25 μL/min. There is a diffusion process before the substrates thoroughly contact enzymes.

To test the flow volume of the process (i.e., dead volume), pNP was pumped to flow through the bioreactor until the concentration of collection was the same as the concentration used. The flow bioreactor was executed by mounting one piece (diameter: 5.5 mm, height: 5 mm) of BGU@OBS. The spiked Surine mixture (glucuronides: 23.8 ppb, internal standard: 4.76 ppb) was pumped to run through the bioreactor at different flow rates (5, 10, 50 μL/min). Each fraction was collected every 250 μL and quenched with 160 μL of solvent with 5% FA in MeOH. The samples were transferred into a bGone Plus plate, centrifuged at 500 g for 1 min, and then diluted using water (the volume of supernatants and water was 200 vs 600 μL) before LC MS analysis. The dead volume was found to be about 1 mL.

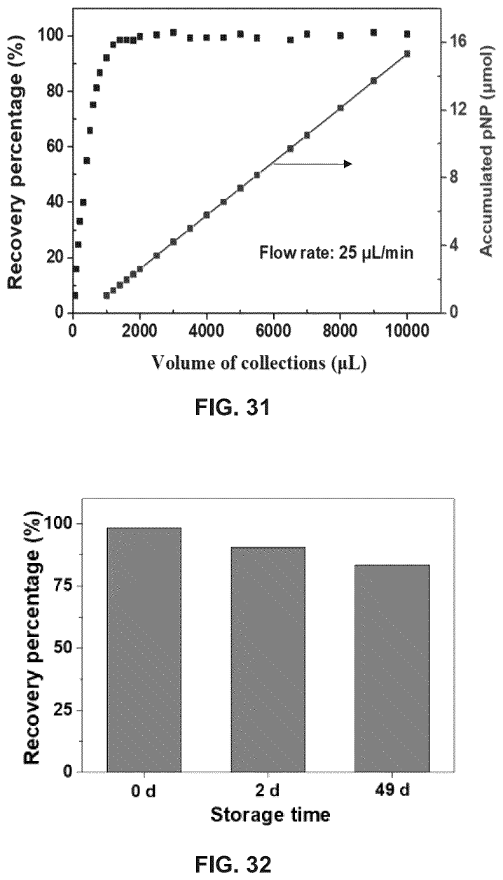

Under the optimized conditions, the flow reaction was carried out with 20 μmol pNPG; around 15.3 μmol pNP (FIG. 31) was accumulated after 6.7 h with 10 mL operation volume. Around 96.2% recovery rate was achieved.

FIG. 32 shows that ˜88% of activity was maintained following storage of the bioreactor for 2 days at 4° C., while ˜83% of the enzyme's activity was maintained after 7 weeks of storage at 4° C., demonstrating good reusability and stability of the OBS-based flow bioreactor.

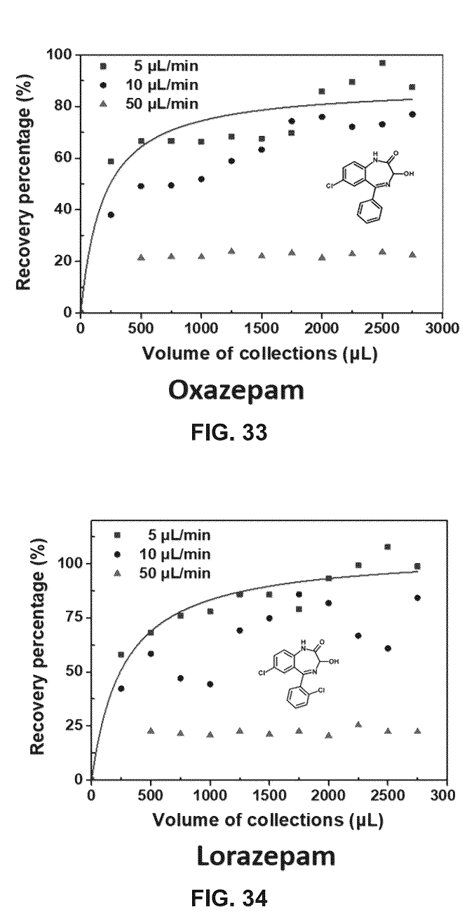

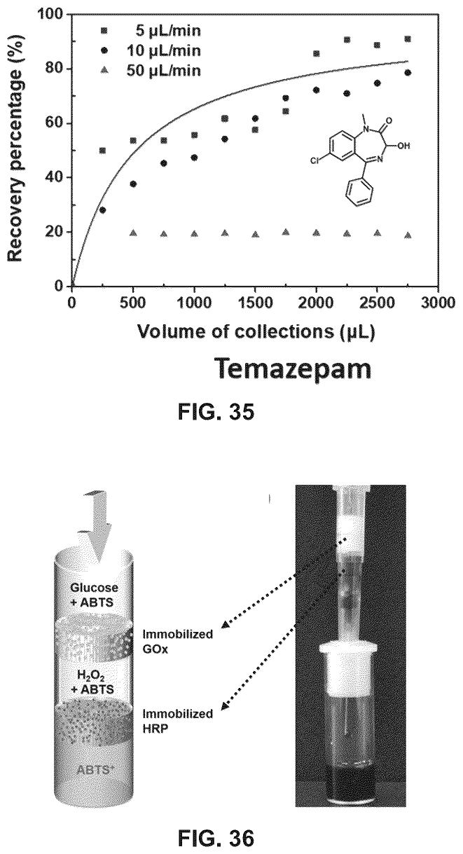

The BGU@OBS flow reactor was applied to perform the hydrolysis of glucuronidated benzodiazepine drugs of abuse in synthetic urine. Three different representative benzodiazepine drugs were utilized: oxazepam, lorazepam, and temazepam. As shown in FIG. 33, FIG. 34, and FIG. 35, the BGU@OBS presented different recovery percentages at different rates. When the flow rate was set to 5 μL/min, the hydrolysis of glucuronides was above 90% within 1.5 mL of flow-through. The hydrolysis process of the BGU@OBS bioreactor was successfully applied for accurate detection of these three glucuronidated drugs, achieving over 90% recovery of glucuronides of glucuronidated benzodiazepines in Surine samples.

### Flowing Reaction for Tandem Catalysis

The system was expanded to multienzyme reactions. Glucose oxidase (GOx) and horse-radish peroxidase (HRP) were readily immobilized on separated OBSs to construct a dual-enzyme reactor, in which GOx@OBS first oxidized glucose to gluconolactone under the production of H₂O₂. The H₂O₂ can then be used by HRP@OBS as a co-substrate for oxidation of the substrate ABTS. The functions of the two enzymes after immobilization were identified by the chromogenic assay. A schematic and photograph of the dual enzyme bioreactor is shown in FIG. 36.

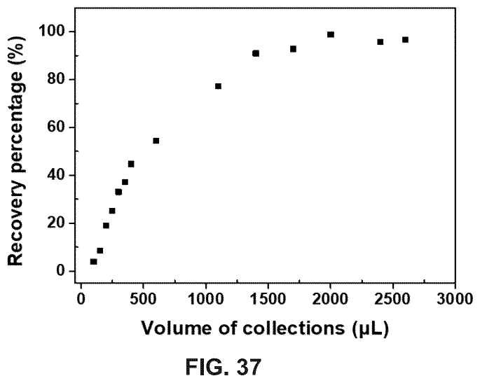

The initial feed solution was clear and the product solution had a green color that was from the ABTS⁺, the product catalyzed by HRP@OBS in the presence of GOx@OBS and D-glucose, which indicated the functionality of the enzymes after immobilization. Similar to the results obtained in the single enzyme BGU-based bioreactor, the recovery percentage reached ˜98% after ˜1.5 mL of flow-through (FIG. 37), which could be explained by the process of substrate diffusion before thorough contact with the enzymes. The result shown in FIG. 37 also implies that the immobilized GOx and HRP had good stability and reusability. Usually, lignin and hemicelluloses in other wood-based supports are degraded by H₂O₂. Interestingly, no apparent deleterious effect was observed, which indicates that the delignified immobilized scaffolds have a predominant advantage when applied in this combined biosystem.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. A cellulose-based scaffold comprising:
delignified bamboo, the delignified bamboo comprising a hierarchical assembly of cellulose fibers and hemicellulose chains, the hierarchical assembly defining an interconnected pore structure including sieve tubes, parenchymal cell lumen, and inner cell wall pores; and
an enzyme immobilized at a surface of the delignified bamboo.

2. The cellulose-based scaffold of claim 1, wherein the enzyme is immobilized at the surface via an alkylamine linkage.

3. The cellulose-based scaffold of claim 1, wherein the interconnected pore structure comprises pores having an average pore size of greater than about 120 μm that comprise from about 5% to about 10% of the total interconnected pore structure, pores having an average pore size of from about 50 μm to about 120 μm that comprise from about 15% to about 25% of the total interconnected pore structure, pores having an average pore size of from about 10 μm to about 50 μm that comprise from about 45% to about 55% of the total interconnected porosity, and pores having an average pore size of less than about 10 μm that comprise from about 20% to about 30% of the total interconnected porosity.

4. The cellulose-based scaffold of claim 1, wherein the delignified bamboo has a density of from about 0.1 g/cm³ to about 0.5 g/cm³.

5. The cellulose-based scaffold of claim 1, wherein the delignified bamboo has a total porosity of from about 75% to about 95%.

6. A flow bioreactor comprising the cellulose-based scaffold of claim 1.

7. The flow bioreactor of claim 6, wherein the flow bioreactor is a multienzyme flow bioreactor.

8. A method for forming a cellulose-based scaffold, the method comprising:
delignifying a portion of a bamboo culm;
oxidizing the surface of the delignified bamboo culm to form a plurality of aldehyde groups on the surface;
contacting the surface with an enzyme, wherein upon the contact an amine of the enzyme reacts with an aldehyde group at the surface to form a Schiff base; and
reducing the Schiff base to form an alkylamine linkage between the enzyme and the surface.

9. The method of claim 8, wherein the step of delignifying the bamboo comprises contacting the portion of the bamboo culm with an oxidizing acidic solution.

10. The method of claim 9, wherein the oxidizing acidic solution comprises an acid and an oxidizing compound.

11. The method of claim 10, wherein the acid and the oxidizing compound comprise only carbon, hydrogen, and oxygen.

12. The method of claim 10, wherein the oxidizing acidic solution comprises acetic acid and hydrogen peroxide.

13. The method of claim 8, wherein the step of oxidizing the surface of the delignified bamboo comprises contacting the delignified bamboo with a periodate oxidation compound.

14. The method of claim 13, wherein the periodate oxidation compound comprises sodium periodate.

15. The method of claim 8, wherein the step of reducing the Schiff base comprises contacting the delignified bamboo with a reducing agent comprising cyanoborohydride, 2-picoline borane, borane diethylamine, or a combination thereof.

16. A method for catalyzing a reaction comprising contacting a first cellulose-based scaffold with a fluid comprising a substrate, the first cellulose-based scaffold comprising first delignified bamboo and a first enzyme immobilized at a surface of the first delignified bamboo, the first delignified bamboo comprising a hierarchical assembly of cellulose fibers and hemicellulose chains, the hierarchical assembly defining an interconnected pore structure including sieve tubes, parenchymal cell lumen, and inner cell wall pores, wherein upon the contact the first enzyme catalyzes a first reaction of the substrate to form a first product within the fluid.

17. The method of claim 16, wherein the first cellulose-based scaffold is retained in a flow bioreactor, the step of contacting comprising flowing the fluid through the flow bioreactor.

18. The method of claim 17, further comprising collecting the fluid containing the first product.

19. The method of claim 16, further comprising following the first reaction, contacting the fluid containing the first product with a second cellulose-based scaffold, the second cellulose-based scaffold comprising second delignified bamboo and a second enzyme immobilized at a surface of the second delignified bamboo, the second delignified bamboo comprising a hierarchical assembly of cellulose fibers and hemicellulose chains, the hierarchical assembly defining an interconnected pore structure including sieve tubes, parenchymal cell lumen, and inner cell wall pores, wherein upon the contact the second enzyme catalyzes a second reaction of the first product to form a second product.

20. The method of claim 19, further comprising collecting the fluid containing the second product.
